(19) United States
(12) Patent Application Publication
(10) Pub. No.: US 2016/0279101 A1
(43) Pub. Date: Sep. 29, 2016

(54) PHARMACEUTICAL COMPOSITION FOR TREATMENT OF PNEUMONIA ASSOCIATED WITH FUSARIUM FUNGUS

(71) Applicants: POLA PHARMA INC., Tokyo (JP); NIHON NOHYAKU CO., LTD., Tokyo (JP)

(72) Inventors: Tsuyoshi Shimamura, Yokohama-shi, Kanagawa (JP); Hiroyasu Koga, Tokyo (JP); Yasuko Nanjoh, Kawachinagano-shi, Osaka (JP)

(21) Appl. No.: 15/037,488

(22) PCT Filed: Nov. 14, 2014
(86) PCT No.: PCT/JP2014/080834

§ 371 Date: May 18, 2016

(30) Foreign Application Priority Data
Nov. 19, 2013 (JP) 2013-239179

(51) Int. Cl. A61K 31/4178; C12Q 1/68

(52) U.S. Cl. CPC A61K 31/4178; C12Q 1/6895; C12Q 2600/112

(57) ABSTRACT

The present invention aims to provide a pharmaceutical composition for pneumonia associated at least with a Fusarium fungus as a causative microorganism. Provided is a pharmaceutical composition for pneumonia associated at least with a Fusarium fungus as a causative microorganism, which pharmaceutical composition includes as an effective component a compound represented by the General Formula (1) below:

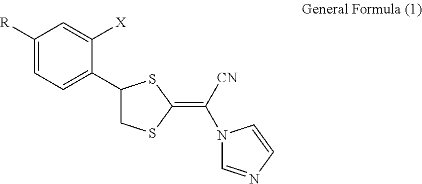

(wherein R represents a halogen atom or hydrogen atom, and X represents a halogen atom).

### TECHNICAL FIELD

The present invention relates to a pharmaceutical composition for pneumonia, more specifically, a pharmaceutical composition for pneumonia associated with a Fusarium fungus or a fungus of genus Fusarium.

### BACKGROUND ART

With the advent of an aging society, and due to increasingly stressful environments and the like, patients with mycotic pneumonia have been increasing in recent years. The cure rate of mycotic pneumonia is basically not high, and the prognosis is especially poor in cases of pneumonia caused by a fungus that is originally not parasitic in human, for example, a Fusarium fungus such as Fusarium oxysporum or Fusarium solani (see, for example, Non-patent Document 1). The Fusarium fungi herein are plant-parasitic fungi which are not susceptible to normal antifungal agents such as terbinafine and bifonazole, and cause lettuce leaf rot, pea root rot and the like. Human infection with these fungi has also been reported recently. Examples of possible mechanisms of such infection include host factors that allow infection with fungi that are originally not infectious to human, that is, low immunity, disturbance of the immune system in the lungs due to complex infection, and the like. In particular, inapparent infection with Trichomonas protozoans and pneumonia caused by such infection, and inapparent infection with Chlamydia intracellular parasites and pneumonia caused by such infection are becoming prevalent in recent years. Thus, disturbance of the immune system by these infections and pneumonia are not negligible as factors that may cause infection with Fusarium fungi. It is said that prevalence of illicit sexual activities is contributing to the prevalence of Trichomonas infection and Chlamydia infection, and patients with these infections may, increase also in the future.

For pneumonia or inapparent infection caused by Chlamydia, a newquinolone, tetracycline or macrolide antibiotic is employed, and, for pneumonia or inapparent infection caused by Trichomonas, metronidazole is employed. However, appearance of strains resistant to these therapeutic agents has been a problem (see, for example, Non-patent Document 2).

In other words, it can be said that the prognosis of pneumonia associated with a fungus that is originally not parasitic in human is poor because there is no reliable therapeutic method for the underlying pneumonia or inapparent infection caused by Chlamydia or Trichomonas.

Moreover, in cases of occurrence of such infection with a fungus that is originally not parasitic in human, pneumonia and inapparent infection by Chlamydia or Trichomonas are often not taken into account. This is because, if such complex infection is taken into account, identification of the microorganisms involved in the pneumonia and selection of the therapeutic agent should be carried out before the initiation of the therapy, and this may often lead to delay of treatment of the fungal or mycotic pneumonia itself. On the other hand, if the complex infection is not taken into account, treatment of the pneumonia is difficult since the disturbance of the immune system continues.

It can be said that, under such circumstances, development of means that enables treatment of mycotic pneumonia with a single drug without taking Chlamydia and Trichomonas into account has been demanded.

On the other hand, treatment of mycotic pneumonia using an antifungal agent has been known (see, for example, Patent Document 1, Patent Document 2 and Patent Document 3). It is also known that luliconazole has excellent antifungal activity against Fusarium oxysporum and Fusarium solani (see, for example, Non-patent Document 3 and Patent Document 4). However, no action of luliconazole on protozoans such as Trichomonas or on intracellular parasites such as Chlamydia has been known at all. Moreover, there is no known pharmaceutical composition, at all, for pneumonia associated with a Fusarium fungus, which pharmaceutical composition comprises as an effective component luliconazole and should be used as a single drug for the treatment without taking association of a Trichomonas protozoan and/or Chlamydia intracellular parasite into account.

### PRECEDING TECHNICAL DOCUMENTS

### Patent Documents

Patent Document 1: Japanese Translated PCT Patent Application Laid-open No. 2004-521102
Patent Document 2: Japanese Translated PCT Patent Application Laid-open No. 2002-514165
Patent Document 3: Japanese Translated PCT Patent Application Laid-open No. 2003-527308
Patent Document 4: WO 2013/047530

### Non-Patent Documents

Non-patent Document 1: Rodriguez-Villalobos H. et. al., Eur. J. Clin. Microbiol. Infect. Dis., 2002 21(2), 149-52
Non-patent Document 2: Pal C, Bandyopadhyay U., Antioxid Redox Signal. 2012, 17(4), 555-82
Non-patent Document 3: Uchida K. et. al., J. Infect. Chemotherap. 2004, 10(4), 216-219

### SUMMARY OF THE INVENTION

### Technical Problem

The present invention was made under such circumstances, and aims to provide a pharmaceutical composition for pneumonia associated at least with a Fusarium fungus as a causative microorganism, which should be used as a single drug for the treatment without taking association of a protozoan(s) such as Trichomonas and/or intracellular parasite(s) such as Chlamydia into account, that is, a pharmaceutical composition for pneumonia associated at least with a Fusarium fungus as a causative microorganism, and also potentially with a protozoan(s) such as Trichomonas and/or intracellular parasite(s) such as Chlamydia as a causative microorganism(s).

### Solution to Problem

In view of these circumstances, the present inventors intensively studied in order to find a pharmaceutical composition for pneumonia associated at least with a Fusarium fungus as a causative microorganism, which should be used as a single drug for the treatment without taking association of a protozoan(s) such as Trichomonas and/or intracellular parasite(s) such as Chlamydia into account. As a result, the present inventors discovered that pharmaceutical compositions comprising as an effective component a compound represented by the General Formula (1) below such as luliconazole or lanoconazole have the above properties, thereby completing the present invention. That is, the present invention is as described below.

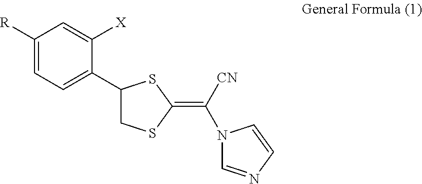

(wherein R represents a halogen atom or hydrogen atom, and X represents a halogen atom).

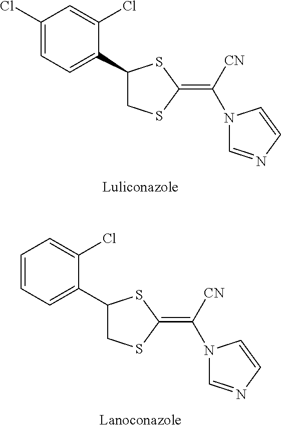 [SMILES: N#C/C(=C1/SC[C@@H](c2ccc(Cl)cc2Cl)S1)n1ccnc1 ; N#C/C(=C1/SCC(c2ccccc2Cl)S1)n1ccnc1]

<1> A pharmaceutical composition for pneumonia associated at least with a Fusarium fungus as a causative microorganism, the pharmaceutical composition comprising as an effective component a compound represented by the General Formula (1).
<2> The pharmaceutical composition for pneumonia according to <1>, wherein the compound represented by the General Formula (1) is luliconazole or lanoconazole.
<3> The pharmaceutical composition for pneumonia according to <1> or <2>, wherein the Fusarium fungus is Fusarium oxysporum and/or Fusarium solani.
<4> The pharmaceutical composition for pneumonia according to any one of <1> to <3>, wherein the pharmaceutical composition for pneumonia is for radical treatment of pneumonia associated at least with a Fusarium fungus as a causative microorganism.
<5> The pharmaceutical composition for pneumonia according to any one of <1> to <4>, wherein the pneumonia is associated with a Fusarium fungus/fungi and also with a protozoan(s) and/or intracellular parasite(s) as causative microorganisms.
<6> A pharmaceutical composition for pneumonia associated at least with a Fusarium fungus as a causative microorganism, and also potentially with a protozoan(s) and/or intracellular parasite(s) as a causative microorganism(s), the pharmaceutical composition comprising as an effective component a compound represented by the General Formula (1):

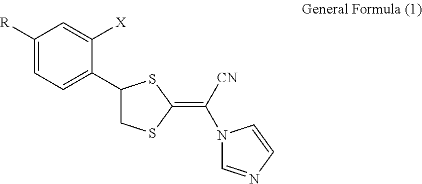

(wherein R represents a halogen atom or hydrogen atom, and X represents a halogen atom).
<7> A system for treatment of pneumonia associated at least with a Fusarium fungus as a causative microorganism, the system comprising:

means for detecting a causative microorganism of pneumonia in a body fluid of a patient with pneumonia;

the pharmaceutical composition for pneumonia according to any one of <1> to <6>; and

means for administering the pharmaceutical composition;

wherein, according to detection of the Fusarium fungus as the causative microorganism of pneumonia in the patient with pneumonia by the detection means, the pharmaceutical composition is administered to the patient with pneumonia by the administration means.
<8> A method for treating pneumonia associated at least with a Fusarium fungus as a causative microorganism, the method comprising: collecting a body fluid from a patient with pneumonia; confirming that a Fusarium fungus is a causative microorganism of the pneumonia; and then administering the pharmaceutical composition according to any one of <1> to <6> without confirming the presence or absence of protozoan infection and/or intracellular parasite infection.

### Advantageous Effects of the Invention

The present invention can provide a pharmaceutical composition for pneumonia associated at least with a Fusarium fungus as a causative microorganism, which should be used as a single drug for the treatment without taking association of a protozoan(s) such as Trichomonas and/or intracellular parasite(s) such as Chlamydia into account, that is, a pharmaceutical composition for pneumonia associated at least with a Fusarium fungus as a causative microorganism, and also potentially with a protozoan(s) such as Trichomonas and/or intracellular parasite(s) such as Chlamydia as a causative microorganism(s).

### BRIEF DESCRIPTION OF THE DRAWINGS

FIG. 1 is a diagram (photographs) illustrating the results of observation of chlamydial inclusion bodies after luliconazole treatment, which observation was carried out by fluorescent staining using a Chlamydia FA reagent “Seiken”. Panel (A) shows the result of observation of chlamydial inclusion bodies after treatment with 8 μg/mL luliconazole. Panel (B) shows the result of observation of chlamydial inclusion bodies after treatment with 16 μg/mL luliconazole. Panel (C) shows the result of observation of chlamydial inclusion bodies after treatment with 32 μg/mL luliconazole. In panel (A) and panel (B), chlamydial inclusion bodies were found as spots stained in apple green. In panel (C), no inclusion body was found.

### DESCRIPTION OF THE EMBODIMENTS

### <1> Compound Represented by General Formula (1)

The pharmaceutical composition of the present invention comprises a compound represented by General Formula (1), and is for pneumonia associated with a Fusarium fungus/fungi.

That is, the pharmaceutical composition of the present invention comprises a compound represented by General Formula (1), and is for pneumonia associated at least with a Fusarium fungus as a causative microorganism.

Examples of the Fusarium fungus include Fusarium oxysporum, Fusarium solani and Fusarium agiaticum, and the pharmaceutical composition is preferably applied to pneumonia associated with Fusarium oxysporum or Fusarium solani, whose frequency of infection is high.

In General Formula (1), the group represented by R is a hydrogen atom or halogen atom, and preferred examples of the halogen atom include a chlorine atom, bromine atom, fluorine atom and iodine atom. The group is especially preferably a hydrogen atom or chlorine atom.

The group represented by X is a halogen atom. Preferred examples of the halogen atom include a chlorine atom, bromine atom, fluorine atom and iodine atom. The group is especially preferably a chlorine atom.

Among the compounds represented by General Formula (1), luliconazole (R═X═Cl; (R)-(−)-(E)-[4-(2,4-dichlorophenyl)-1,3-dithiolan-2-ylidene]-1-imidazolylacetonitrile) and lanoconazole (R═H, X═Cl; 4-(2-chlorophenyl)-1,3-dithiolan-2-ylidene-1-imidazolylacetonitrile) are preferred, and luliconazole is especially preferred. Such compound components not only suppress the growth of protozoans such as Trichomonas and intracellular parasites such as Chlamydia, but also suppress the growth of Fusarium fungi such as Fusarium oxysporum and Fusarium solani.

These compounds can be synthesized according to the method described in JP 60-218387 A. That is, 1-cyanomethylimidazole is reacted with carbon disulfide to obtain a compound (III), which is then reacted with a compound of General Formula (II) having leaving groups, to thereby obtain a compound represented by General Formula (1). Preferred examples of the leaving groups include methanesulfonyloxy, benzenesulfonyloxy, p-toluenesulfonyloxy, and halogen atoms.

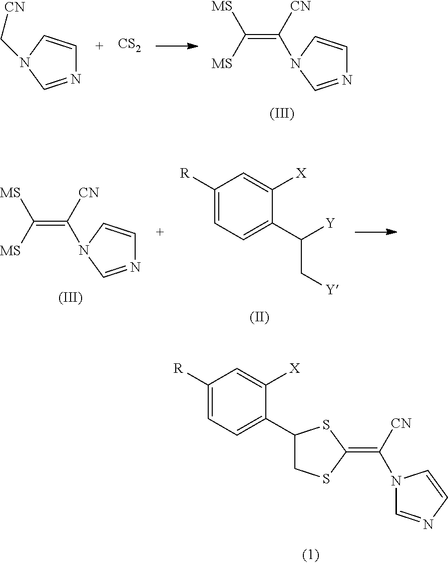

(wherein Y and Y′ each represents a leaving group such as methanesulfonyloxy, benzenesulfonyloxy, p-toluenesulfonyloxy or a halogen atom; and M represents an alkali metal).

In order for the compound represented by General Formula (1) to exert antiprotozoal action, antifungal action and anti-intracellular parasite action, the compound represented by General Formula (1) may be added usually at 0.5 to 80% by mass, more preferably at 1 to 80% by mass, still more preferably at 1 to 60% by mass with respect to the total amount of the pharmaceutical composition.

### <2> Pharmaceutical Composition of Present Invention

The pharmaceutical composition of the present invention may contain an arbitrary component for formulation other than the compound represented by the General Formula (1). The component for formulation is preferably contained as the remaining part other than the compound represented by the General Formula (1). The total amount of the component for formulation is usually 20 to 99.5% by mass, preferably 20 to 99% by mass, more preferably 40 to 99% by mass with respect to the total amount of the pharmaceutical composition of the present invention.

In cases of a tablet, preferred examples of the component for formulation include vehicles such as lactose and croscarmellose; alkaline agents such as sodium carbonate and sodium hydrogen carbonate; acidic agents such as citric acid, lactic acid and tartaric acid; coating agents such as ethyl cellulose, hydroxypropyl methylcellulose and triethyl citrate; binders such as gum arabic; disintegrators such as starch, crystallized cellulose and hydroxypropyl cellulose; sugar coatings such as sucrose and maltitol; surfactants such as POE hydrogenated castor oil and POE sorbitan fatty acid esters; plasticizers such as triethyl citrate, caprylic acid/capric acid monoglyceride and diethylene glycol monoethyl ether; and lubricants such as magnesium stearate and talc.

The dosage form of the pharmaceutical composition of the present invention may be an injection solution. Examples of the dosage form as an injection solution that may be employed include injection solutions containing a solubilized clathrate, and injection solutions in which an effective component is carried by liposomes, niosomes, fine lipid particles, self-assembled emulsion or the like. Preferred examples of components suitable for such dosage forms include phospholipids such as cyclodextrin, phosphatidylcholine, phosphatidic acid, phosphatidylinositol, phosphatidylglycerol and phosphatidylserine; self-assembling agents such as acylated tripeptide; polyols such as glycerol, propylene glycol and 1,3-butanediol; and surfactants such as POE hydrogenated castor oil and POE sorbitan fatty acid esters; which may be modified. For adjustment of the osmotic pressure, an electrolyte such as sodium chloride may also be added.

Alternatively, the compound represented by General Formula (1) may be made into fine powder to provide an inhalation formulation that is to be directly inhaled into the lungs.

Alternatively, the pharmaceutical composition may be in the form of a suppository. In cases of a suppository, examples of formulation components that may be used for the formulation include hydrocarbons such as vaseline, solid paraffin, microcrystalline wax and liquid paraffin; esters such as olive oil, castor oil, Witepsol, carnauba wax, Japan wax and beeswax; higher alcohols such as stearyl alcohol, cetostearyl alcohol, oleyl alcohol and benzyl alcohol; and surfactants such as monoglyceryl stearate, monoglyceryl oleate and sorbitan fatty acid esters.

The pharmaceutical composition of the present invention can be produced according to a conventional method using the compound represented by the General Formula (1) and the arbitrary component(s) for formulation.

The pharmaceutical composition of the present invention may be used either as a formulation which is absorbed through the gastrointestinal tract and the mucosa, or as a formulation which is absorbed without passing through the gastrointestinal tract or the mucosa. The pharmaceutical composition is especially preferably used as a formulation which is absorbed without passing through the gastrointestinal tract. Since, unlike metronidazole, the compound of the present invention represented by General Formula (1) does not show strong mutagenicity, the compound can be safely administered in such modes.

A preferred mode of the application of the compound may be arbitrarily selected in consideration of the body weight, age, sex and symptoms of the patient, and the like. Usually, in adults, the pharmaceutical composition may be orally or parenterally (as an injection solution, nasal drops, suppository, inhalant or the like) administered once or several times per several days such that the dose of the compound represented by General Formula (1) is 0.1 to 10 g, and such treatment may be carried out for about 1 week to 3 months.

The compound represented by General Formula (1) has not only antifungal action against Fusarium fungi, but also antiprotozoal action against protozoans such as Trichomonas, and anti-intracellular parasite action against intracellular parasites such as Chlamydia. The pharmaceutical composition of the present invention was invented based on such discovery by the present inventors.

That is, the pharmaceutical composition of the present invention may be applied to pneumonia associated with an intracellular parasite(s), protozoan(s) and/or Fusarium fungus/fungi as a pathogen(s) (for example, pneumonia diagnosed as having been caused by an intracellular parasite(s), protozoan(s) and/or Fusarium fungus/fungi as a pathogen(s)).

The “pharmaceutical composition of the present invention for pneumonia associated with a protozoan(s) as a pathogen(s)” may be applied to pneumonia associated with a protozoan(s) as a pathogen(s), and to pneumonia associated both with a protozoan(s) and with a Fusarium fungus/fungi and/or intracellular parasite(s) as pathogens. Under the present circumstances, where a protozoan(s) as well as a Fusarium fungus/fungi and/or intracellular parasite(s) often coexist, and secondary infection or the like with a protozoan(s) frequently occurs, it is also preferred to apply the “pharmaceutical composition of the present invention for pneumonia associated with a protozoan(s) as a pathogen(s)” to pneumonia associated with a Fusarium fungus/fungi and/or intracellular parasite(s) as a pathogen(s), from the viewpoint of suppressing potential infection with the protozoan(s) and preventing the secondary infection. The application to pneumonia associated with a Fusarium fungus/fungi and/or intracellular parasite(s) as a pathogen(s) for such a purpose is also included within the scope of the present invention.

The “pharmaceutical composition of the present invention for pneumonia associated with intracellular parasite(s) as a pathogen(s)” may be applied to pneumonia associated with intracellular parasite(s) as a pathogen(s), and to pneumonia associated both with intracellular parasite(s) and with a Fusarium fungus/fungi and/or a protozoan(s) as pathogens. Under the present circumstances, where intracellular parasite(s) as well as a Fusarium fungus/fungi and/or a protozoan(s) often coexist, and secondary infection or the like with intracellular parasite(s) frequently occurs, it is also preferred to apply the “pharmaceutical composition of the present invention for pneumonia associated with intracellular parasite(s) as a pathogen(s)” to pneumonia associated with a Fusarium fungus/fungi and/or a protozoan(s) as a pathogen(s), from the viewpoint of suppressing potential infection with the intracellular parasite(s) and preventing the secondary infection. The application to pneumonia associated with a Fusarium fungus/fungi and/or a protozoan(s) as a pathogen(s) for such a purpose is also included within the scope of the present invention.

The “pharmaceutical composition of the present invention for pneumonia associated with a Fusarium fungus/fungi as a pathogen(s)” may be applied to pneumonia associated with a Fusarium fungus/fungi as a pathogen(s), and to pneumonia associated both with a Fusarium fungus/fungi and with a protozoan(s) and/or intracellular parasite(s) as pathogens. Under the present circumstances, where a Fusarium fungus/fungi as well as a protozoan(s) and/or intracellular parasite(s) often coexist, and secondary infection or the like with a Fusarium fungus/fungi frequently occurs, it is also preferred to apply the “pharmaceutical composition of the present invention for pneumonia associated with a Fusarium fungus/fungi as a pathogen(s)” to pneumonia associated with a protozoan(s) and/or intracellular parasite(s) as a pathogen(s), from the viewpoint of suppressing potential infection with the fungus/fungi and preventing the secondary infection. The application to pneumonia associated with a protozoan(s) and/or intracellular parasite(s) as a pathogen(s) for such a purpose is also included within the scope of the present invention.

The “pharmaceutical composition of the present invention for pneumonia associated with a Fusarium fungus/fungi, protozoan(s) and intracellular parasite(s) as a pathogen(s)” can be applied not only to pneumonia associated with a Fusarium fungus/fungi, protozoan(s) and intracellular parasite(s) as pathogens, but also to pneumonia associated with a protozoan(s) as a pathogen(s), pneumonia associated with a Fusarium fungus/fungi as a pathogen(s) and pneumonia associated with an intracellular parasite(s) as a pathogen(s) from the viewpoint of suppressing potential infection with the Fusarium fungus/fungi, protozoan(s) and/or intracellular parasite(s) and preventing secondary infection therewith. The application to pneumonia associated with a Fusarium fungus/fungi as a pathogen(s), pneumonia associated with a protozoan(s) as a pathogen(s) or pneumonia associated with an intracellular parasite(s) as a pathogen(s) for such a purpose is also included within the scope of the present invention.

The pharmaceutical composition of the present invention for pneumonia associated with a Fusarium fungus/fungi as a pathogen(s) has a property that enables, in cases where the association of the Fusarium fungus/fungi with the pneumonia is apparent, treatment of the pneumonia until complete cure using the pharmaceutical composition of the present invention alone without examining association of protozoans such as Trichomonas and intracellular parasites such as Chlamydia. This is because, even under the coexistence of Trichomonas and/or Chlamydia, these pathogenic microorganisms can be eliminated at the same time, and there is therefore only a very low possibility of survival of the Fusarium fungus/fungi behind these pathogenic microorganisms.

“Complete cure of pneumonia” herein means a state where the causative microorganism cannot be detected even 1 month after completion of administration of the agent.

The pharmaceutical composition of the present invention is used through the following steps.

<Step 1> The causative microorganism is collected from a body fluid collected from a patient with pneumonia, and subjected to culture, if desired. The obtained microorganism cells are subjected to judgment of whether a Fusarium fungus is present or not using detection means.

<Step 2> In cases where the presence of the Fusarium fungus was found in Step 1, the pharmaceutical composition of the present invention is administered by administration means.

Preferred examples of the means for judging the presence of the Fusarium fungus herein include methods such as real-time PCR. Preferred examples of the administration means include means such as infusion, means such as injection, and means such as tablets. The presence of Trichomonas, Chlamydia and the like does not need to be examined at this time. This is because compounds represented by General Formula (1) such as luliconazole and lanoconazole have actions to kill these pathogens, and, by treating the Fusarium mycosis, these infections can also be treated. Thus, treatment can be carried out such that the Fusarium fungus is not hidden behind the affected area of Trichomonas infection or the affected area of Chlamydia infection, and complete cure of the pneumonia can therefore be expected.

### <3> System for Treatment of Pneumonia

These operations can be carried out as a series of operations, and such a flow of operations is referred to as the system in the present invention. In the system of the present invention, each step as a constituting unit of the system may also be carried out by an artificial operation. That is, the user of the system may also carry out means such as detection and administration.

Each step as a constituting unit of the system may also be carried out by automated means. The “means for administering the pharmaceutical composition” also includes means for giving instruction to administer the pharmaceutical composition or for displaying to administer the pharmaceutical composition.

That is, the system of the present invention is as follows.

A system for treatment of pneumonia associated at least with a Fusarium fungus as a causative microorganism, the system comprising:

means for detecting a causative microorganism of pneumonia in a body fluid of a patient with pneumonia;

a pharmaceutical composition for pneumonia associated at least with a Fusarium fungus as a causative microorganism, the pharmaceutical composition comprising as an effective component a compound represented by General Formula (1); and

means for administering the pharmaceutical composition;

wherein, according to detection of the Fusarium fungus as the causative microorganism of pneumonia in the patient with pneumonia by the detection means, the pharmaceutical composition is administered to the patient with pneumonia by the administration means.

### <4> Method for Treatment of Pneumonia

The present invention further relates to a method for treating pneumonia associated at least with a Fusarium fungus as a causative microorganism, the method comprising: collecting a body fluid from a patient with pneumonia; confirming that a Fusarium fungus is a causative microorganism of the pneumonia; and then administering a pharmaceutical composition for pneumonia associated at least with a Fusarium fungus as a causative microorganism without confirming the presence or absence of protozoan infection and/or intracellular parasite infection, the pharmaceutical composition comprising as an effective component a compound represented by General Formula (1).

### EXAMPLES

The present invention is described below in more detail by way of Examples.

### Example 1

Among the compounds represented by General Formula (1), luliconazole was selected for investigation of the effect on Trichomonas vaginalis. That is, 5×10⁶ cells of clinically isolated Trichomonas vaginalis were seeded in Trichomonas Medium F, manufactured by Fuji Pharma Industrial Co., Ltd., which contains neutral red as a marker (6.5 mL, contained in a tube). Preculture was performed for 72 hours (preculture). After confirming that the Trichomonas has grown to actively produce acid and to thereby cause changing of the color of neutral red to yellow, 100 μL of the obtained preculture liquid was added to Trichomonas Medium F to be used for main culture. To resulting mixture, 0.5 mL of each test liquid was further added. The number of Trichomonas cells in the preculture liquid at this time was 1.5×10⁵ cells/mL. Three types of test liquids, that is, solutions of luliconazole in 10% methanol/saline solution at luliconazole concentrations of 200 μM (final concentration, 35.2 μM), 100 μM (final concentration, 17.6 μM) and 50 μM (final concentration, 8.8 μM), were provided. A control was prepared by adding 0.5 mL of a vehicle as a test liquid. As the vehicle, 10% methanol/saline solution (final concentration, 0 μM) was used. After the addition, each resulting mixture was stirred well, and culture was carried out at 37° C. for 72 hours. Thereafter, the color was judged, and the condition of Trichomonas cells was observed under an inverted microscope. The results are shown in Table 1. The results indicate that 8.8 μM luliconazole inhibited the growth of Trichomonas. In other words, luliconazole was found to be a substance except metronidazole that can be clinically applied and can inhibit the growth of Trichomonas. It can also be seen that the minimum inhibitory concentration (MIC) is about 8.8 μM.

TABLE 1

| Final | | |
|---|---|---|
| concentration | Color | Result of observation under the microscope |
| 35.2 μM | Red | No Trichomonas cell was found |
| 17.6 μM | Red | No Trichomonas cell was found |
| 8.8 μM | Yellow | A small number of Trichomonas cells were |
| | | found |
| 0 μM | Yellow | A large number of Trichomonas cells were |
| | | found |

### Example 2

The same study as in Example 1 was carried out except that lanoconazole was used instead of luliconazole. As a result, similarly to luliconazole, lanoconazole was found to inhibit the growth of Trichomonas. Thus, lanoconazole was found to be a substance except metronidazole that can be clinically applied and can inhibit the growth of Trichomonas. It can also be seen that the minimum inhibitory concentration (MIC) is about 17.6 μM.

TABLE 2

| Final | | |
|---|---|---|
| concentration | Color | Result of observation under the microscope |
| 35.2 μM | Red | No Trichomonas cell was found |
| 17.6 μM | Red | A small number of Trichomonas cells were |
| | | found |
| 8.8 μM | Yellow | A large number of Trichomonas cells were |
| | | found |
| 0 μM | Yellow | A large number of Trichomonas cells were |
| | | found |

### Example 3

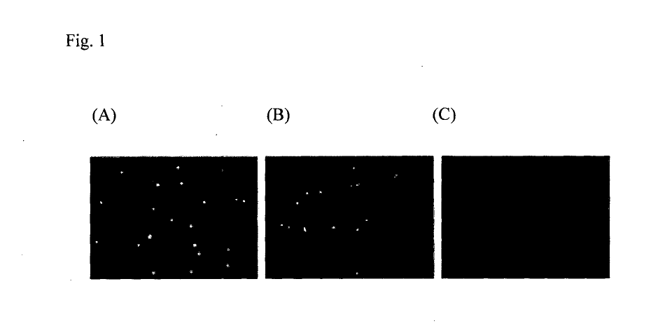

Using Chlamydia trachomatis (D/UW3/Cx), the anti-intracellular parasite action was examined. That is, Chlamydia trachomatis was cultured using HeLa 229 cells as a host in the presence of a 2-fold dilution series of 8 to 64 μg/mL luliconazole. The culture was carried out using, as a medium, MEM supplemented with 1 μg/mL cyclohexamide and 8% heat-inactivated FBS, at 37° C. under 5% carbon dioxide for 72 hours. Thereafter, inclusion bodies were fluorescently stained in apple green using a Chlamydia FA reagent “Seiken” (manufactured by Denka Seiken Co., Ltd.), and observed under a fluorescence microscope. The results obtained for the samples at luliconazole concentrations of 8, 16 and 32 μg/mL are shown in FIG. 1. By this, it can be seen that the MIC of luliconazole against Chlamydia trachomatis is 32 μg/mL.

### Example 4

The MICs of each type of antifungal agent against Fusarium oxysporum and Fusarium solani were determined. That is, each fungus was cultured at 35° C. for 72 hours using, as a medium, “RPMI 1640/MOPS liquid medium supplemented with 10% Alamar Blue (registered trademark) (pH 7.0)” (see Shinobu Ishigaki et al., The Journal of the Japanese Association for Infectious Diseases, vol. 74(3) (2000) pp. 221-230) according to the micro-liquid dilution method (Alamar Blue assay), which is in accordance with the CLSI standardization and the Japanese Society for Medical Mycology method. Thereafter, O.D. was measured for investigating color change of the oxidation-reduction indicator Alamar Blue, to determine the minimum inhibitory concentration (MIC). That is, the growth inhibition rate (IC) was determined based on the O.D. value, and the minimum concentration of the agent at which an IC of not less than 80% was achieved was determined as MIC. Two strains of Fusarium oxysporum (clinically separated strains; Teikyo University Institute of Medical Mycology), and 10 strains of Fusarium solani (clinically separated strains; Institute of Dermatology (Thailand) and Teikyo University Institute of Medical Mycology) were used. The antifungal agents used in the study are shown in Table 3. The determined MICs are shown in Table 4. It can be seen from these results that, among the antifungal agents tested, only luliconazole and lanoconazole are effective for Fusarium fungi including Fusarium oxysporum and Fusarium solani.

TABLE 3

| Type | Agent name (abbreviation) | Indication |
|---|---|---|
| Polyene- | Amphotericin B (AMPB) | Deep mycosis |
| compounds | | |
| Triazole- | Voriconazole (VCZ) | |
| compounds | Itraconazole (ITZ) | |
| | Fluconazole (FCZ) | |
| | Efinaconazole (ECZ) | Superficial |
| Imidazole- | Luliconazole (LLCZ) | mycosis |
| compounds | Lanoconazole (LCZ) | |
| | Bifonazole (BFZ) | |
| | Ketoconazole (KCZ) | |
| | Clotrimazole (CTZ) | |
| | Miconazole nitrate (MCZ) | |
| Morpholine- | Amorolfine hydrochloride | |
| compounds | (AMO) | |
| Allylamine- | Terbinafine (TBF) | |
| compounds | | |

TABLE 4

| Agent | F. solani (10) | F. oxysporum (2) |
|---|---|---|
| LLCZ | 0.063 | 0.022 |
| | 0.016-0.063 | 0.016-0.031 |
| LCZ | 0.25 | 0.090 |
| | 0.063-0.25 | 0.063-0.13 |
| BFZ | >4 | >4 |
| | >4 | >4 |
| KCZ | >4 | >4 |
| | 4->4 | >4 |
| CTZ | >4 | >4 |
| | >4 | >4 |
| MCZ | >4 | >4 |
| | 4->4 | >4 |
| EFCZ | ND | 0.5 |
| | | 0.25-1 |
| VCZ | >4 | >4 |
| | 1->4 | >4 |
| ITZ | >4 | >4 |
| | >4 | >4 |
| FCZ | >4 | >4 |
| | >4 | >4 |
| AMO | >4 | >4 |
| | >4 | >4 |
| TBF | >4 | 4 |
| | 4->4 | 2->4 |
| AMB | 4 | >4 |
| | 2-4 | 4->4 |

### Example 5

According to the following formulation, tablets for oral administration were prepared. That is, the part A was subjected to granulation, and the resulting granules were made into tablets, followed by coating of the tablets by spraying of ethyl cellulose (coating agent) and triethyl citrate (plasticizer) dissolved in ethanol. Thereafter, the coated tablets were dried by blowing warm air at 40° C., to prepare tablets for oral administration.

TABLE 5
(A)

| | | |
|---|---|---|
| Starch | 15 | parts by mass |
| Crystallized cellulose | 15 | parts by mass |
| Lactose | 20 | parts by mass |
| Luliconazole | 40 | parts by mass |
| Lactic acid | 0.5 | part by mass |
| Hydroxypropyl cellulose | 0.5 | part by mass |

(Coating agent)

| | | |
|---|---|---|
| Ethyl cellulose | 8 | parts by mass |
| Triethyl citrate | 1 | part by mass |

### Example 6

In the same manner as in Example 5, tablets were prepared by processing the following components.

TABLE 6
(A)

| | | |
|---|---|---|
| Starch | 15 | parts by mass |
| Crystallized cellulose | 15 | parts by mass |
| Lactose | 20 | parts by mass |
| Lanoconazole | 40 | parts by mass |
| Lactic acid | 0.5 | part by mass |
| Hydroxypropyl cellulose | 0.5 | part by mass |

(Coating agent)

| | | |
|---|---|---|
| Ethyl cellulose | 8 | parts by mass |
| Triethyl citrate | 1 | part by mass |

### INDUSTRIAL APPLICABILITY

The present invention can be applied to pharmaceuticals.

1. A pharmaceutical composition for pneumonia associated at least with a Fusarium fungus as a causative microorganism, said pharmaceutical composition comprising as an effective component a compound represented by the General Formula (1) below: 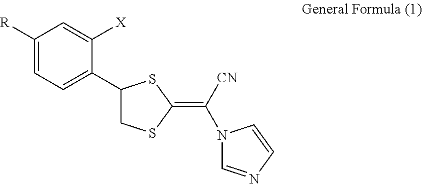
wherein R represents a halogen atom or hydrogen atom, and X represents a halogen atom.

2. The pharmaceutical composition for pneumonia according to claim 1, wherein said compound represented by the General Formula (1) is luliconazole or lanoconazole.

3. The pharmaceutical composition for pneumonia according to claim 1, wherein said Fusarium fungus is Fusarium oxysporum and/or Fusarium solani.

4. The pharmaceutical composition for pneumonia according to claim 1, wherein said pharmaceutical composition for pneumonia is for radical treatment of pneumonia associated at least with a Fusarium fungus as a causative microorganism.

5. The pharmaceutical composition for pneumonia according to claim 1, wherein said pneumonia is associated with a Fusarium fungus/fungi and also with a protozoan(s) and/or intracellular parasite(s) as causative microorganisms.

6. The pharmaceutical composition for pneumonia according to claim 1, wherein said pneumonia is also potentially associated with a protozoan(s) and/or intracellular parasite(s) as a causative microorganism(s)
(wherein R represents a halogen atom or hydrogen atom, and X represents a halogen atom).

7. A system for treatment of pneumonia associated at least with a Fusarium fungus as a causative microorganism, said system comprising:
means for detecting a causative microorganism of pneumonia in a body fluid of a patient with pneumonia;
the pharmaceutical composition for pneumonia according to claim 1; and
means for administering said pharmaceutical composition;
wherein, according to detection of said Fusarium fungus as the causative microorganism of pneumonia in said patient with pneumonia by said detection means, said pharmaceutical composition is administered to said patient with pneumonia by said administration means.

8. A method for treating pneumonia associated at least with a Fusarium fungus as a causative microorganism, comprising:
administering to a subject in need thereof a pharmaceutical composition comprising as an effective component a compound represented by the General Formula (1) below: 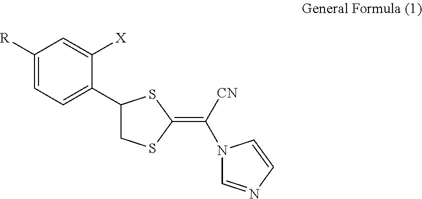
(wherein R represents a halogen atom or hydrogen atom, and X represents a halogen atom).

9. The method according to claim 8, wherein said compound represented by the General Formula (1) is luliconazole or lanoconazole.

10. The method according to claim 8, wherein said Fusarium fungus is Fusarium oxysporum and/or Fusarium solani.

11. The method according to claim 8, wherein said method is for radical treatment of pneumonia associated at least with a Fusarium fungus as a causative microorganism.

12. The method according to claim 8, wherein said pneumonia is associated with a Fusarium fungus/fungi and also with a protozoan(s) and/or intracellular parasite(s) as causative microorganisms.

13. The method according to claim 8, wherein said pneumonia is also potentially associated with a protozoan(s) and/or intracellular parasite(s) as a causative microorganism(s).
